(19) United States
(12) Patent Application Publication
(10) Pub. No.: US 2022/0192935 A1
(43) Pub. Date: Jun. 23, 2022

(54) COMPOSITION FOR A TOPICAL APPLICATION ON THE SKIN

(71) Applicants: Thomas SCHMIDTS, Giessen (DE); Frank RUNKEL, Buseck-Oppenrod (DE); Michael MERZHÄUSER, Wetzlar (DE); Biodermic Health & Beauty GmbH & Co. KG, Gießen (DE)

(72) Inventors: Thomas Schmidts, Uberlingen (DE); Frank Runkel, Buseck-Oppenrod (DE); Michael Merzhäuser, Wetzlar (DE)

(21) Appl. No.: 17/594,337

(22) PCT Filed: Apr. 14, 2020
(86) PCT No.: PCT/EP2020/060377

§ 371 Date: Oct. 12, 2021

(30) Foreign Application Priority Data
Apr. 12, 2019 (DE) 10 2019 109 828.9

(51) Int. Cl. A61K 8/04; A61K 8/06; A61K 8/37; A61Q 19/00; A61Q 17/00

(52) U.S. Cl. CPC A61K 8/046; A61K 8/062; A61Q 17/005; A61K 8/375; A61Q 19/007; A61K 8/064

(57) ABSTRACT

A composition for topical application to the skin, comprising at least one humectant, at least one consistency regulator, and at least one emulsifier, wherein the emulsifier includes a fatty acid esterified with a hydroxyl group, wherein the fatty acid has at least one carbon chain of 18 or fewer carbon atoms. The emulsifier can include ethers, polymers, amides, amines, or sulfates.

### CROSS-REFERENCE TO RELATED APPLICATIONS

This application is a filing under 35 U.S.C. 371 of International Application No. PCT/EP2020/060377, filed Apr. 14, 2020, entitled “Composition for a Topical Application to the Skin,” which claims priority to German Patent Application No. 10 2019 109 828.9, filed Apr. 12, 2019, which applications are incorporated by reference herein in their entirety.

### FIELD OF THE DISCLOSURE

According to a first aspect, the invention relates to a composition for topical application to the skin as claimed in claim 1.

### BACKGROUND

The surface of the skin features a specific microbial flora consisting of bacteria and fungi which each colonize different regions of the skin in varying numbers. This microbial flora, also referred to as resident “skin flora” or “skin microflora”, represent parts of what is known as the microbiome and contributes significantly to protecting the skin and hence the organism as a whole from pathogens.

In terms of bacterial pathogens, the most important representatives of the resident skin flora are Corynebacterium, Streptococcus, Staphylococcus and Propionibacterium. These make up approximately 90% of the total number of bacteria on the skin. Concerning the skin fungi, Malassezia yeast species play a significant role. Important strains of these species are for example Malassezia furfur, Malassezia sympodialis, Malassezia globosa and Malassezia restricta. At approximately 90%, they make up the greatest part of the fungi on the skin.

Current investigations have found that a dysregulation of the above organisms on the skin is critically responsible for common human skin disorders such as for example acne, rosacea, perioral dermatitis and Pityriasis versicolor, pityrosporum folliculitis and also for atopic and seborrhoeic eczema, or at least is involved significantly in the pathogenesis thereof.

Besides an individual dysregulation of the skin flora, additional cofactors are required for the occurrence and manifestation, and for the progression, of such disorders. Among these cofactors, the ingredients of externally applied skincare products appear to play a key role in particular. The ingredients most frequently used in cosmetics or other external agents are oils or emollients, consistency regulators and emulsifiers.

Malassezia yeasts feed on skin fats and thus are often found in areas of the skin which contain high numbers of sebaceous glands. The disorders mentioned also manifest in these areas. Malassezia and various bacteria possess a multitude of fat-cleaving enzymes which are secreted onto the skin surface. The enzymes secreted by the microorganisms also cleave the fats and oils present in common cosmetics and skincare products.

This process forms breakdown products which can massively influence the equilibrium of the skin flora. The formation of solid breakdown products is especially problematic since these can clog the pores of the skin. In addition, some species can feed on common cosmetic ingredients which are supplied to the skin, which in turn can lead to overpopulation of the respective species and can disrupt the equilibrium of the skin flora.

When using customary and commercially available cosmetic products, additional lipid sources are also provided which can be metabolized by Malassezia. This can result in amplified growth of the Malassezia species on the skin. The growth increase can be observed especially when applying products which contain natural oils, fats and/or long-chain fatty acid esters.

The common components of commercially available skincare and body care products consequently result in the occurrence or exacerbation of skin disorders. Common components of these care products in particular include the following substances: In terms of oils/emollients, synthetic long-chain fatty acid esters, natural substances such as for example grapeseed oil or hydrocarbon-based substances such as for example paraffin are commonly used.

In terms of consistency regulators, use is generally made of synthetic esters (for example cetyl palmitate), natural waxes (for example beeswax), fatty alcohols (for example cetearyl alcohol) and/or gel formers.

Emulsifiers known are especially long-chain synthetic fatty acid esters.

### SUMMARY

The object of the invention is to overcome the above disadvantages of the prior art and to provide a composition for topical application to the skin which counteracts the occurrence or exacerbation of skin disorders. The composition is to have skincare, skin protection, cleansing and cosmetic properties and at the same time is to be simple and cheap to produce.

Main features of the invention are given in claims 1, 5 and 10. Preferred configurations are described by claims 2 to 4, 6 to 9 and 11 to 16.

### DETAILED DESCRIPTION

Unless stated otherwise, the chemical substances, substance groups or components mentioned herein are referred to in accordance with the “International Nomenclature of Cosmetic Ingredients” (INCI).

In a first aspect, the invention relates to a composition for topical application to the skin, comprising at least one humectant, at least one consistency regulator, and at least one emulsifier, wherein the emulsifier includes a fatty acid esterified with a hydroxyl group, wherein the fatty acid has at least one carbon chain of 18 or fewer carbon atoms. When referring to the chain length, only the carbon atoms present in the chain are taken into account. The fact that the individual carbon atoms of a carbon chain consisting of n carbon atoms may possess other atoms such as for example hydrogen atoms or even other side groups is obvious and is not taken into account in the nomenclature.

A fatty acid esterified with a hydroxyl group is also referred to as a fatty acid ester. According to the invention, according to a first aspect of the invention, as emulsifier, medium-chain or short-chain fatty acid esters or derivatives of such fatty acid esters can accordingly be provided.

The term carbon chain refers to a structural element of a compound which comprises two or more carbon-carbon bonds. The carbon atoms are therefore “chained” together—these are also referred to as hydrocarbon chains. Examples of molecules having such carbon chains are caproic acid (6 carbon atoms), caprylic acid (8 carbon atoms), capric acid (10 carbon atoms) and lauric acid (12 carbon atoms).

In the context of this application, an emulsifier is understood to be an interface-active auxiliary which is used to mix and to stabilize two immiscible or at least poorly miscible liquids to give a finely dispersed mixture (what is known as an emulsion). Emulsifiers are often also referred to as surfactants. Any structural or functional differences which may exist between emulsifiers and surfactants depending on definition are not taken into account in the present application.

It has surprisingly been found that a composition which includes at least one emulsifier, which includes at least one fatty acid which is esterified with a hydroxyl group and has at least one carbon chain of 18 or fewer carbon atoms, interacts with the skin flora only to a minor degree, if at all. This prevents excessive growth of individual pathogens, as a result of which disruption to the equilibrium of the microflora on the skin can be counteracted. Furthermore, the fatty acid fraction is insoluble and can precipitate out, which in the case of large molecules can in turn lead to clogging of the skin pores. For molecules with more than 18 carbon atoms in a carbon chain, the risk of clogging skin pores therefore also increases. It has been found that the use in particular of substances having fatty acid moieties having a carbon chain of 12 or fewer carbon atoms significantly reduces the risk of clogging the skin pores.

The surprising positive effect of the above-described substances or substance groups on the growth of the microorganisms of the skin flora has been confirmed by investigations on microorganisms of the skin flora. Specifically, this involved analyzing the growth of the following species: Corynebacterium minutissimum, Streptococcus mitis, Staphylococcus epidermidis, Staphylococcus galinarum, Propionibacterium acnes, and Malassezia furfur, Malassezia sympodialis, Malassezia globosa and Malassezia restricta. The positive effect can be attributed to the fact that the microorganisms present on the skin cannot make use of the substances investigated.

According to an advantageous development, it is provided that the emulsifier includes a fatty acid esterified with a hydroxyl group, wherein the fatty acid has at least one carbon chain of 16 or fewer carbon atoms. It is in particular provided that the carbon chain comprises 12 or fewer carbon atoms.

The use of one or more of the aforementioned substances or substance groups as a component of the composition according to the invention (alone or in combination) has proven to be particularly advantageous because they do not have any growth-promoting influence on the organisms of the skin flora. Investigations on Corynebacterium minutissimum, Streptococcus mitis, Staphylococcus epidermidis, Staphylococcus galinarum, Propionibacterium acnes, and Malassezia furfur, Malassezia sympodialis, Malassezia globosa and Malassezia restricta have confirmed this effect.

Further advantages emerge from the preferred embodiment according to which the at least one emulsifier is selected from the group comprising hydroxy fatty acid esters, sugar esters and polyglycerol esters. In this case, the following substances or substance groups may in particular be provided:
Macrogol 40 glycerol hydroxystearate
PEG-40 hydrogenated castor oil
Sucrose palmitate
Sucrose laurate
Sucrose stearate
Polyglyceryl-3 Caprate
Polyglyceryl-3 Dicitrate/Stearate
Polyglyceryl-4 Laurate
Polyglyceryl-10 Laurate
Polyglyceryl-6 Caprylate
Polyglyceryl-4 Caprate
Polyglyceryl-3 Caprylate/Caprate/Succinate
Sorbitan laurate
Dilauryl Citrate.

Macrogol 40 glycerol hydroxystearate is a nonionic emulsifier which can be used as a solubilizer for water-insoluble components. Sucrose palmitate, sucrose laurate sucrose stearate, Polyglyceryl-3 Caprate, Polyglyceryl-3 Dicitrate/Stearate, Polyglyceryl-4 Laurate, Polyglyceryl-6 Caprylate, Polyglyceryl-4 Caprate, Polyglyceryl-3 Caprylate/Caprate/Succinate, and sorbitan laurate are emulsifiers with refatting and moisturizing properties. They are especially suitable as emulsifiers for oil-in-water emulsions. Moreover, they have skincare properties, just like Polyglyceryl-10 Laurate and Dilauryl Citrate. PEG-40 hydrogenated castor oil is used as a solubilizer for active ingredients, fragrance oils and colorants, and also as a humectant and hence as a base for creams. Polyglyceryl-3 Caprate and Polyglyceryl-3 Dicitrate/Stearate contain polymerized glycerin. They are used to improve the uniform distribution of skincare products when applied and enable the formation of finely dispersed mixtures of oil and water. Polyglyceryl-3 Dicitrate/Stearate includes a derivative of stearate, that is to say a saturated fatty acid the fatty acid moiety of which has a carbon chain of 18 carbon atoms.

The length of the carbon chain is particularly relevant when the emulsifier is a fatty acid ester. In a further aspect, the invention relates to a composition for topical application to the skin, comprising at least one humectant, at least one consistency regulator, and at least one emulsifier, wherein the emulsifier is selected from the group comprising ethers, polymers, amides, amines and sulfates.

If the emulsifier is an ether, a polymer, an amide, an amine or a sulfate, the growth of the microorganisms on the skin is inhibited. It has thus been found that a composition which includes at least one emulsifier selected from the group comprising ethers, polymers, amides, amines and sulfates only interacts with the skin flora to a minor extent, if at all. This prevents excessive growth of individual pathogens, as a result of which disruption to the equilibrium of the microflora on the skin can be counteracted.

According to a development of the invention, it can also be provided that the at least one emulsifier is an ether, selected from the group comprising PEG ethers and alkyl polyglycosides. In particular, the emulsifier can be selected from the following substances or substance groups:
Ceteareth-25
Ceteth-20
Oleth-20
Laureth-20
Isoceteth-20
Laureth-23
Steareth-21
Steareth-20
Cetearyl glucoside.

Ceteth indicates a polyethylene glycol ether of cetyl alcohol. Correspondingly, Oleth indicates the polyethylene glycol ether of oleyl alcohol. Ceteareth-25, Ceteth-20, Oleth-20, Laureth-20, Laureth-23, Isoceteth-20, Steareth-21 and Steareth-20 are used as emulsifiers for improving the uniform distribution of skincare products when applied. In addition, they enable the formation of finely dispersed mixtures of oil and water. Cetearyl glucoside is an emulsifier based on glucose and coconut oil. In addition to its function as an emulsifier, cetearyl glucoside has particularly good moisturizing properties.

The positive effect of these emulsifiers in terms of their influence on the growth of organisms of the skin flora has been confirmed by investigations on Corynebacterium minutissimum, Streptococcus mitis, Staphylococcus epidermidis, Staphylococcus galinarum, Propionibacterium acnes, and Malassezia furfur, Malassezia sympodialis, Malassezia globosa and Malassezia restricta. No growth-promoting influence was detected. The substances mentioned are therefore suitable as emulsifier in the composition according to the invention.

It is moreover conceivable that the at least one emulsifier is a polymer, selected from the group comprising
Cetyl PEG/PPG-10/1/Dimethicone
Bis-PEG/PPG-16/16 PEG/PPG-16/16 Dimethicone
Bis-PEG/PPG-20/5 PEG/PPG-20/5 Dimethicone
Methoxy PEG/PPG-25/4 Dimethicone
Poloxamer 407
Acrylates/C10-30 Alkyl Acrylate Crosspolymer.

Dimethicone protects the skin from external influences and thus keeps it in a smooth and supple condition. Cetyl PEG/PPG-10/1/Dimethicone is a copolymer of cetyl dimethicone (polydimethylsiloxane) and alkoxylated dimethicone. Poloxamers are block copolymers of ethylene oxide and propylene oxide. As emulsifiers, they are used to improve the uniform distribution of skincare products when applied. In addition, they enable the formation of finely dispersed mixtures of oil and water.

This advantageous property of the abovementioned substances can be observed in particular in the case of the following skin microorganisms: Staphylococcus epidermidis, Staphylococcus galinarum, Corynebacterium minutissimum, Streptococcus mitis and Propionibacterium acnes, and Malassezia furfur, Malassezia sympodialis, Malassezia globosa and Malassezia restricta.

It may be advantageous when the at least one emulsifier is an amide or amine, selected from the group comprising
Sodium Cocoamphoacetate WAS 32%.
Capryl/Capramidopropyl Betaine
Cocamidopropyl Betaine WAS 48%
Cocamidopropyl Betaine
Undecylenamidopropyl Betaine
Disodium Cocoamphodiacetate
Palmitamidopropyltrimonium Chloride
Distearyldimonium Chloride
Disodium/Sodium Cocoyl Glutamate.

These emulsifiers were investigated in terms of their influence on the growth of the organisms of the skin flora. No growth-promoting influence was detected. The substances mentioned are therefore suitable as emulsifier in the composition according to the invention.

Sodium Cocoamphoacetate WAS 32% is an amphoacetate and hence an amide. The carbon chain of the fatty acid moiety of Sodium Cocoamphoacetate WAS 32% has 12 carbon atoms. Sodium Cocoamphoacetate WAS 32% is also a relatively mild emulsifier which only interacts with the skin flora to a minor degree, if at all. It stabilizes foaming compositions, in that it increases the surface tension of the liquid surrounding the individual foam bubbles. Sodium Cocoamphoacetate WAS 32% serves to give shape and volume to hair.

Cocamidopropyl Betaine WAS 48% (CAPB) is an emulsifier selected from the group comprising amides and amines. It exhibits amphoteric behavior in neutral solutions, and thus exhibits different material properties depending on the reaction partner. For example, CAPB behaves as a cationic emulsifier in acidic solutions and in alkaline solutions behaves as an anionic emulsifier. CAPB additionally regulates viscosity and ensures uniform distribution of the composition when applied to the skin. It has cleansing and antistatic effects, in that the electric charge at the skin surface is neutralized. It can additionally be used to improve the foam quality of the composition. Moreover, CAPB has antiseptic properties.

Capryl/Capramidopropyl Betaine, Cocamidopropyl Betaine, Undecylenamidopropyl Betaine, Disodium Cocoamphodiacetate, Palmitamidopropyltrimonium Chloride, Distearyldimonium Chloride and Disodium/Sodium Cocoyl Glutamate are viscosity-regulating substances which ensure a uniform distribution of the composition when applied to the skin. They additionally have cleansing and antistatic effects, thus reducing static electricity, in that the electric charge at the skin surface is neutralized. They can additionally be used to improve the foam quality of the composition. Cocamidopropyl Betaine and Disodium/Sodium Cocoyl Glutamate moreover have antiseptic properties. The good compatibility of these two substances with the mucous membranes additionally favors use in compositions which are to be used on infants.

It is furthermore conceivable that the at least one emulsifier is a sulfate selected from the group comprising Sodium Coco-Sulfate, Sodium Laureth Sulfate and Magnesium Sulfate Heptahydrate. Sodium Coco-Sulfate (SCS) and Sodium Laureth Sulfate (SLS) generate lather and have advantageous thickening and conditioning properties. They are a suitable replacement for anionic surfactants. Magnesium Sulfate Heptahydrate.

The absence of breakdown or the absence of conversion of the individual substances of the composition according to the invention by the microorganisms of the skin flora moreover has the advantageous effect that no breakdown products can be formed which would otherwise cause clogging of the skin pores.

It can furthermore be provided that the at least one emulsifier is a wool wax. Wool wax is a purified, waxy and anhydrous substance obtained from the wool of sheep. Wool wax is water-repellent and hence protects the skin from drying out.

The emulsifiers mentioned above were investigated in terms of their influence on the growth of the organisms of the skin flora. No growth-promoting influence was detected. The substances are therefore suitable as emulsifier in the composition according to the invention.

In a further aspect, the invention relates to a composition for topical application to the skin, comprising at least one humectant, at least one consistency regulator, and at least one emulsifier, wherein the emulsifier is selected from the group comprising ethers, polymers, amides, amines and sulfates and/or wherein the emulsifier includes a fatty acid esterified with a hydroxyl group, wherein the fatty acid has at least one carbon chain of 18 or fewer carbon atoms.

It has surprisingly been found that a composition which includes at least one emulsifier, which includes at least one fatty acid which is esterified with a hydroxyl group, wherein the fatty acid has at least one carbon chain of 18 or fewer carbon atoms, interacts with the skin flora only to a minor degree, if at all. This prevents excessive growth of individual pathogens, as a result of which disruption to the equilibrium of the microflora on the skin can be counteracted. Furthermore, the fatty acid fraction is insoluble and can precipitate out, which in the case of large molecules can in turn lead to clogging of the skin pores. For molecules with fatty acid moieties having more than 18 carbon atoms in a carbon chain, the risk of clogging the skin pores therefore also increases. It has been found that in particular the use of fatty acid esters with fatty acid moieties having a carbon chain of 12 or fewer carbon atoms significantly reduces the risk of clogging of the skin pores.

The surprising positive effect of the above-described substances or substance groups on the growth of the microorganisms of the skin flora has been confirmed by investigations on microorganisms of the skin flora. Specifically, this involved analyzing the growth of the following species: Corynebacterium minutissimum, Streptococcus mitis, Staphylococcus epidermidis, Staphylococcus galinarum, Propionibacterium acnes, and Malassezia furfur, Malassezia sympodialis, Malassezia globosa and Malassezia restricta. The positive effect can be attributed to the fact that the microorganisms present on the skin cannot break down such substances.

If the emulsifier is an ether, a polymer, an amide, an amine or a sulfate, the growth of the microorganisms on the skin is inhibited. It has thus been found that a composition which includes at least one emulsifier selected from the group comprising ethers, polymers, amides, amines and sulfates only interacts with the skin flora to a minor extent, if at all. This can be attributed to the fact that the microorganisms present on the skin cannot break down such molecules. This prevents excessive growth of individual pathogens, as a result of which disruption to the equilibrium of the microflora on the skin can be counteracted.

It may also be provided that the consistency regulator is selected from the group comprising the following substances or substance groups:
xanthan gum
cellulose derivatives (e.g. carmellose sodium)
carbomers
Acrylamide/Sodium Acryloyldimethyl Taurate Copolymer
Hydroxyethyl Acrylate/Sodium Acryloyldimethyl Taurate Copolymer
Sodium Acrylate/Acryloyldimethyltaurate/Dimethylacrylamide Crosspolymer
Hydroxyethyl Acrylate/Sodium Acryloyldimethyl Taurate Copolymer & Polyisobutene & PEG-7 Trimethylolpropane Coconut Ether
Sodium Acrylate/Acryloyldimethyltaurate/Dimethylacrylamide Crosspolymer
Cocamide
capric acid
Sorbitan Sesquicaprylate
Isostearamide MIPA
Glyceryl Laurate
Laureth-4
fatty alcohols having at least one carbon chain of 12 or fewer carbon atoms.

Consistency regulators ensure good stability and good consistency of cosmetic skincare products.

Xanthan gum is a naturally occurring polysaccharide which promotes the imparting of consistency to a composition and improves the product stability. Carmellose sodium is a member of the carboxymethyl celluloses. Carboxymethyl celluloses are derivatives of cellulose in which some of the hydroxyl groups are linked as ether with a carboxymethyl group. Carbomers are members of the polyacrylic acids. Similarly to xanthan gum, both substances have properties in cosmetics promoting the imparting of consistency to a composition. Sorbitan Sesquicaprylate, Palmitamidopropyltrimonium Chloride, Isostearamide MIPA, Glyceryl Laurate and Laureth-4 are wash-active and viscosity-influencing substances which promote a uniform distribution of the composition on the skin.

Capric acid is a fatty acid which improves product consistency. Isostearamide MIPA, Palmitamidopropyltrimonium Chloride and Laureth-4 moreover have particularly good antistatic properties. Acrylates are polymers or copolymers based on acrylic acid or other acrylic esters. Taurates are N-acyl derivatives, esters or salts of taurine. The term copolymers refers to polymers composed of two or more different base units.

With respect to the fatty alcohols, it is provided that the carbon chain of the fatty alcohol respectively present in the composition comprises 12 or fewer carbon atoms. If this is a secondary or tertiary fatty alcohol, it is provided that both or all three carbon chains each comprise 12 or fewer carbon atoms. Such fatty alcohols only interact with the skin flora to a minor degree, if at all. This prevents individual pathogens from growing excessively on the skin. In this way, disruption to the equilibrium of the microflora on the skin can be counteracted.

It may also be advantageous for the composition to comprise at least one emollient. Emollients are substances which influence the degree of roughness or suppleness of the skin. They can make the skin appear softer and smoother overall.

In this case the composition can comprise at least one emollient, which is selected from the group comprising the following substances or substance groups:
Paraffin
Polyisobutene
squalane
silicone oil
Cetyl Dimethicone
Stearyl Dimethicone
squalene
Cyclopentasiloxane
Octyldodecanol
Caprylic/Capric Triglyceride.

The abovementioned emollients are based on hydrocarbons or alcohols and therefore have no influence on the growth of the skin microflora.

It has surprisingly been found that emollients having a fatty acid moiety the carbon chain of which comprises more than 18 carbon atoms lead to increased Malassezia growth. The substances mentioned above are consequently suitable for use as emollients in the composition according to the invention, provided that they have a fatty acid moiety having a carbon chain of 18 or fewer carbon atoms. Preference is given to using emollients having a fatty acid moiety having a carbon chain of 12 or fewer carbon atoms.

Octyldodecanol is an organic compound from the substance class of the alcohols and is used as an additive for the masking of odors and as a fragrance. The longer carbon chain of octyldodecanol consists of 12 carbon atoms.

However, according to a particularly preferred embodiment it is provided that the emollient has a fatty acid moiety having a carbon chain of 12 or fewer carbon atoms. It can in particular be provided here that the emollient is Cetearyl Isononanoate.

An embodiment is also conceivable in which the composition comprises at least one solubilizer, the solubilizer being selected from the group of polyethylene glycols. In this case, the following substances or substance groups are in particular provided:
PEG 300
PEG 350
PEG 550.

The pathogens tested exhibit only minor growth, if any, after addition of PEG. PEG 300, 350 and 550 are therefore particularly well suited as a solubilizing ingredient in the composition according to the invention.

PEG or polyethylene glycol is an aliphatic polyether. The indices 300, 350 and 500 in this case indicate the average molecular mass of the polymer. One of the particular properties of the polyethylene glycols is their water solubility: They are miscible with water in any ratio. Because of this good water solubility, polyethylene glycols are preferably used as solvent.

According to a preferred embodiment, it is additionally provided that the humectant is selected from the group comprising:
glycols and polyhydric alcohols (e.g. Pentylene Glycol or glycerol)
moisturizing factors (e.g. Sodium Lactate, Lactic Acid, magnesium sulfate, Betaine, Ectoin, Zinc PCA, Urea or constituents of the “Natural Moisturizing Factor” of the skin).

The humectants mentioned demonstrably have no growth-promoting influence on the organisms of the skin flora. However, they can be used to prevent the skin from drying out because they bind added water.

Glycols are dihydric alcohols which are derived from ethylene glycol. Besides moisture-binding properties, Pentylene Glycol and glycerol (also called glycerin) also possess antimicrobial and antibacterial properties. Moisturizing factors are substances which act as an emollient in the skin. Sodium Lactate regulates the physiological pH together with other substances as constituent of the skin's own acid mantle. The slightly acidic pH of the acid mantle keeps the microbial composition of the skin in physiological equilibrium. Glycerol primarily has water-binding properties.

According to a particularly preferred embodiment, it is provided that the composition does not contain any of the following substances or substance groups: Isopropyl Myristate; Isopropyl Palmitate; Decyl oleate; Ethyl oleate; Oleyl oleate; grapeseed oil; olive oil; avocado oil; almond oil; refined evening primrose oil; wheatgerm oil; rapeseed oil; peanut oil; soybean oil; sesame oil; jojoba oil; linseed oil; castor oil; Cetyl Palmitate; beeswax; carnauba wax; shea butter, Cetearyl Alcohol; Stearyl Alcohol; Cetyl Alcohol; Glyceryl Stearate; PEG-40 Stearate; Glyceryl Oleate; Tween 80; Span 80; Methyl Glucose Isostearate; Polyglyceryl-3 Oleate; Tween 40; Glyceryl Stearate Citrate; Polyglyceryl-6 Stearate; Polyglyceryl-6 Behenate; palmitic acid; oleic acid; stearic acid.

An advantageous development provides that the composition is a cream, an ointment, a foam, a gel or a lotion. A composition in such a form can be packaged and dispensed in a simple manner. In addition, ointments, creams, lotions, gels and foam can be easily applied to the skin and distributed.

According to a preferred configuration, the invention furthermore proposes that the composition is an oil-in-water emulsion (O/W emulsion) or a water-in-oil emulsion (W/O emulsion). O/W or W/O emulsions can be applied to the skin particularly easily and guarantee an optimal distribution of all ingredients.

According to a further embodiment, the composition can in particular be present in sunscreen formulations comprising chemical or mineral UV-filtering substances. Useful UV-filtering substances in particular include the following substances: Titanium Dioxide, Zinc Oxide, silicon dioxide, Aluminum Stearate, Alumina, Octocrylene, Bis-Ethylhexyloxyphenol Methoxyphenyl Triazine, Drometrizole Trisiloxane, Terephthalylidene Dicamphor Sulfonic Acid, Ethylhexyl Triazone, Butyl Methoxydibenzoylmethane, Diethylamino Hydroxybenzoyl Hexyl Benzoate, Diethylhexyl Butamido Triazone, Phenylbenzimidazole Sulfonic Acid.

It can additionally be provided that the composition according to the invention is used for skin protection, for skin regeneration, for soothing the skin or as an anti-ageing agent. In this case, the composition can additionally contain one or more of the following substances:
active ingredients from the group of vitamins (e.g: vitamins A, B, C, D or E)
Tocopheryl Acetate
Tocopherol
Panthenol
cell protection factors (e.g. Ecotin or Betaine)
antioxidant, skin-protecting, anti-inflammatory, skin-regenerating active ingredients or plant extracts (such as e.g. tea extract or acerola extract)
skin-conditioning bacterial or fungal lysates or ferments
skin-tightening substances (hyaluronic acid and derivatives thereof)
active ingredients for collagen and connective tissue synthesis (e.g. phytosphingosine and derivatives thereof, peptides)
components of the skin's natural acid mantle, the skin's natural film of fat and of the natural moisture film of the skin.

Tocopheryl Acetate and Tocopherol are antioxidants. Tocopheryl Acetate is an acetic ester of vitamin E. It inhibits oxygen-caused oxidation processes and hence the degradation and deterioration of other ingredients of the composition. Tocopherol, also a derivative of vitamin E, is an umbrella term for all tocopherols having vitamin E activity. Vitamin E is a member of the fat-soluble vitamins. It builds up skin protection and promotes the regeneration of the skin.

Panthenol has skincare properties. It increases the moisture-retention capacity and improves the elasticity of the skin. It also promotes the new formation of skin cells and thus contributes to regeneration. Furthermore, dexpanthenol also has itch-relieving and anti-inflammatory properties and properties promoting wound-healing.

It can moreover be provided that the composition has at least a first phase (A) and a second phase (B). According to a development, the composition can also have a third phase (C). A subdivision of the composition into different phases is advantageous in particular for the production, since the subdivision into phases facilitates uniform mixing of the ingredients.

Further features, details and advantages of the invention emerge from the wording of the claims and also from the following description of exemplary embodiments.

### Exemplary Embodiment 1

According to a first exemplary embodiment, the composition contains the substances specified in table 1.

TABLE 1
Ingredients of a composition of the
invention according to example 1

| Phase | INCI nomenclature | Amount [%] |
|---|---|---|
| A | Aqua Dest. | 68.00 |
| | Pentylene Glycol | 5.00 |
| | Magnesium Sulfate Heptahydrate | 0.10 |
| | Glycerin | 2.50 |
| | Panthenol | 1.35 |
| | Sodium Lactate | 0.10 |
| | Ectoin | 1.00 |
| B | Tocopheryl Acetate | 0.20 |
| | Tocopherol | 0.05 |
| | Carbomer | 0.20 |
| | Octyldodecanol | 8.00 |
| | Caprylic/Capric Triglyceride | 10.50 |
| | Polyglyceryl-3 Dicitrate/Stearate | 3.00 |
| C | Lactic Acid, pH 5.3-5.5 | q.s |

In all exemplary embodiments, water is present as the first solvent. Here, “Aqua dest.” is to be understood as meaning a form (not defined in more detail) of ultrapure water, that is to say for example water that has been distilled once or multiple times, “HPLC-grade” water etc.

Polyglyceryl-3 Dicitrate/Stearate is a polyglycerol ester and thus is an emulsifier which includes a fatty acid esterified with a hydroxyl group, the fatty acid having at least one carbon chain of 18 or fewer carbon atoms. It has refatting and moisturizing properties. It is especially suitable as emulsifier for oil-in-water emulsions. In addition, this emulsifier has skincare properties. Polyglyceryl-3 Dicitrate/Stearate additionally contains polymerized glycerol and thus serves to improve the uniform distribution of skincare products when applied. Polyglyceryl-3 Dicitrate/Stearate includes a derivative of stearic acid, i.e. a saturated fatty acid the fatty acid moiety of which has a carbon chain of 18 carbon atoms.

It has surprisingly been found that a composition which includes at least one emulsifier, which includes at least one fatty acid which is esterified with a hydroxyl group, wherein the fatty acid has at least one carbon chain of 18 or fewer carbon atoms, interacts with the skin flora only to a minor degree, if at all. This prevents excessive growth of individual pathogens, as a result of which disruption to the equilibrium of the microflora on the skin can be counteracted. Furthermore, the fatty acid fraction is insoluble and can precipitate out, which in the case of large molecules can in turn lead to clogging of the skin pores. For molecules with fatty acid moieties having more than 18 carbon atoms in a carbon chain, the risk of clogging skin pores therefore also increases.

Caprylic/Capric Triglyceride is an emulsifier from the group of polymers. Magnesium Sulfate Heptahydrate is an emulsifier from the group of sulfates and generates lather and has advantageous thickening and conditioning properties. Magnesium Sulfate Heptahydrate is a suitable replacement for anionic surfactants.

If the emulsifier is an ether, a polymer, an amide, an amine or a sulfate, the growth of the microorganisms on the skin is additionally inhibited. A composition which includes at least one emulsifier selected from the group comprising ethers, polymers, amides, amines and sulfates only interacts with the skin flora to a minor extent, if at all.

Like all glycols, Pentylene Glycol is derived from ethylene glycol. Moisturizing factors such as Sodium Lactate and Ectoin are substances which act as emollients in the skin. Sodium Lactate regulates the physiological pH together with other substances as constituent of the skin's own acid mantle. The slightly acidic pH of the acid mantle keeps the microbial composition of the skin in physiological equilibrium. Glycerol (Glycerin) has primarily water-binding properties.

Panthenol has skincare properties. It increases the moisture-retention capacity and improves the elasticity of the skin by increasing the rate of lipid synthesis. It also promotes the new formation of skin cells and thus contributes to regeneration. Furthermore, panthenol also has itch-relieving and anti-inflammatory properties and properties promoting wound-healing.

Tocopheryl Acetate and Tocopherol are antioxidants. Tocopheryl Acetate is an acetic ester of vitamin E. It inhibits oxygen-caused oxidation processes and hence the degradation and deterioration of other ingredients of the composition. Tocopherol, also a derivative of vitamin E, is an umbrella term for all tocopherols having vitamin E activity. Vitamin E is a member of the fat-soluble vitamins. It builds up cell protection and promotes the regeneration of the skin.

Octyldodecanol is an organic compound from the substance class of the alcohols and is used as an additive for the masking of odors and as a fragrance.

To produce the emulsion shown by way of example in table 1, first of all the substances of phase A are dissolved or mixed in the sequence specified. Next, or in parallel therewith, phase B is dissolved or mixed in the sequence specified and added to phase A. This is followed by setting the pH to 5.3 to 5.5 using lactic acid (phase C).

The mixture of phases A, B and C is homogenized at approximately 12 000 rpm (revolutions per minute). Lastly, the emulsion is stirred until smooth. The final composition is consequently a homogeneous emulsion of phases A, B and C.

All steps for producing the emulsion according to example 1 are preferably carried out at room temperature.

### Exemplary Embodiment 2

According to a second exemplary embodiment, the composition contains the substances specified in table 2.

TABLE 2
Ingredients of a composition of the
invention according to example 2

| Phase | INCI nomenclature | Amount [%] |
|---|---|---|
| A | Aqua Dest. | 3.00 |
| | Ectoin | 0.10 |
| | Glycerin | 30.20 |
| | Panthenol | 1.35 |
| | Sodium Lactate | 0.10 |
| | Sucrose palmitate | 2.00 |
| B | Tocopheryl Acetate | 0.10 |
| | Xanthan gum | 0.10 |
| | Tocopherol | 0.05 |
| | Octyldodecanol | 20.00 |
| | Caprylic/Capric Triglyceride | 40.00 |

Sucrose palmitate is a hydroxy fatty acid ester and hence is an emulsifier which includes a fatty acid esterified with a hydroxyl group, wherein the fatty acid has at least one carbon chain consisting of 18 or fewer carbon atoms. It has refatting and moisturizing properties and is highly suitable as an emulsifier for oil-in-water emulsions. In addition, it has skincare properties. Sucrose palmitate includes a derivative of palmitic acid, i.e. a saturated fatty acid the fatty acid moiety of which has a carbon chain consisting of 16 carbon atoms.

The positive effect that results from the use of an emulsifier which includes a fatty acid esterified with a hydroxyl group, wherein the fatty acid has at least one carbon chain consisting of 18 carbon atoms (example 1), is additionally amplified by the use of a corresponding emulsifier having a carbon chain consisting of 16 carbon atoms.

Caprylic/Capric Triglyceride is an emulsifier from the group of polymers. If the emulsifier is selected from the group of polymers, the growth rate of the microorganisms on the skin is additionally reduced.

A composition which includes at least one emulsifier selected from the group comprising ethers, polymers, amides, amines and sulfates only interacts with the skin flora to a minor extent, if at all. This prevents excessive growth of individual pathogens, as a result of which disruption to the equilibrium of the microflora on the skin can be counteracted.

Glycerol (Glycerin) has primarily water-binding properties.

Panthenol has skincare properties. It increases the moisture-retention capacity and improves the elasticity of the skin by increasing the rate of lipid synthesis. It also promotes the new formation of skin cells and thus contributes to regeneration. Furthermore, panthenol also has itch-relieving and anti-inflammatory properties and properties promoting wound-healing.

Moisturizing factors such as Sodium Lactate and Ectoin are substances which act as emollients in the skin. Sodium Lactate regulates the physiological pH together with other substances as constituent of the skin's own acid mantle. The slightly acidic pH of the acid mantle keeps the microbial composition of the skin in physiological equilibrium.

Tocopheryl Acetate and Tocopherol are antioxidants. Tocopheryl Acetate is an acetic ester of vitamin E. It inhibits oxygen-caused oxidation processes and hence the degradation and deterioration of other ingredients of the composition. Tocopherol, also a derivative of vitamin E, is an umbrella term for all tocopherols having vitamin E activity. Vitamin E is a member of the fat-soluble vitamins. It builds up skin protection and promotes the regeneration of the skin.

Xanthan gum is a naturally occurring polysaccharide which promotes the imparting of consistency to a composition and improves the product stability.

Octyldodecanol is an organic compound from the substance class of the alcohols and is used as an additive for the masking of odors and as a fragrance.

To produce the composition shown by way of example in table 2, the listed substances of phase A are mixed together in the sequence specified. In parallel with this, the listed substances of phase B are mixed together in the sequence specified. Both phases are then combined. The final composition is a homogeneous emulsion of phases A and B.

### Exemplary Embodiment 3

According to a third exemplary embodiment, the composition contains the substances specified in table 3.

TABLE 3
Ingredients of a composition of the
invention according to example 3

| Phase | INCI nomenclature | Amount [%] |
|---|---|---|
| A | Aqua Dest. | 3.00 |
| | Ectoin | 0.10 |
| | Glycerin | 30.20 |
| | Panthenol | 1.35 |
| | Sodium Lactate | 0.10 |
| | Sucrose laurate | 3.00 |
| B | Tocopheryl Acetate | 0.10 |
| | Tocopherol | 0.05 |
| | Octyldodecanol | 20.00 |
| | Xanthan gum | 0.10 |
| | Caprylic/Capric Triglyceride | 40.00 |

Sucrose laurate is a sugar ester and hence is an emulsifier which includes a fatty acid esterified with a hydroxyl group, wherein the fatty acid has at least one carbon chain consisting of 18 or fewer carbon atoms. It has refatting and moisturizing properties and is highly suitable as an emulsifier for oil-in-water emulsions. In addition, it has skincare properties. Sucrose laurate includes a derivative of lauric acid, i.e. a saturated fatty acid the fatty acid moiety of which has a carbon chain consisting of 12 carbon atoms.

The positive effect that results from the use of an emulsifier which includes a fatty acid esterified with a hydroxyl group, wherein the fatty acid has at least one carbon chain consisting of 18 or 16 carbon atoms (examples 1 and 2), is additionally amplified by the use of a corresponding emulsifier having a carbon chain consisting of 12 carbon atoms.

Caprylic/Capric Triglyceride is an emulsifier from the group of polymers. If the emulsifier is selected from the group of polymers, the growth of the microorganisms on the skin is additionally reduced.

A composition which includes at least one emulsifier selected from the group comprising ethers, polymers, amides, amines and sulfates only interacts with the skin flora to a minor extent, if at all. This prevents excessive growth of individual pathogens, as a result of which disruption to the equilibrium of the microflora on the skin can be counteracted.

Glycerol (Glycerin) has primarily water-binding properties.

Panthenol has skincare properties. It increases the moisture-retention capacity and improves the elasticity of the skin by increasing the rate of lipid synthesis. It also promotes the new formation of skin cells and thus contributes to regeneration. Furthermore, panthenol also has itch-relieving and anti-inflammatory properties and properties promoting wound-healing.

Moisturizing factors such as Sodium Lactate and Ectoin are substances which act as emollients in the skin. Sodium Lactate regulates the physiological pH together with other substances as constituent of the skin's own acid mantle. The slightly acidic pH of the acid mantle keeps the microbial composition of the skin in physiological equilibrium.

Tocopheryl Acetate and Tocopherol are antioxidants. Tocopheryl Acetate is an acetic ester of vitamin E. It inhibits oxygen-caused oxidation processes and hence the degradation and deterioration of other ingredients of the composition. Tocopherol, also a derivative of vitamin E, is an umbrella term for all tocopherols having vitamin E activity. Vitamin E is a member of the fat-soluble vitamins. It builds up skin protection and promotes the regeneration of the skin.

Xanthan gum is a naturally occurring polysaccharide which promotes the imparting of consistency to a composition and improves the product stability.

Octyldodecanol is an organic compound from the substance class of the alcohols and is used as an additive for the masking of odors and as a fragrance.

The emulsion according to exemplary embodiment 3 is produced in an analogous manner to exemplary embodiment 2.

### Exemplary Embodiment 4

According to a fourth exemplary embodiment, the composition contains the substances specified in table 4.

TABLE 4
Ingredients of a composition of the
invention according to example 4

| Phase | INCI nomenclature | Amount [%] |
|---|---|---|
| A | Aqua Dest. | 78.70 |
| | Pentylene Glycol | 5.00 |
| | Cetearyl Glucoside | 1.00 |
| | Glycerin | 2.50 |
| | Panthenol | 1.35 |
| | Sodium Lactate | 0.10 |
| | Ectoin | 0.50 |
| | Polyglyceryl-3 Caprate | 0.50 |
| B | Tocopheryl Acetate | 0.20 |
| | Tocopherol | 0.05 |
| | Xanthan gum | 0.10 |
| | Octyldodecanol | 5.00 |
| | Caprylic/Capric Triglyceride | 5.00 |
| C | Lactic Acid, pH 5.3-5.5 | q.s |

Polyglyceryl-3 Caprate is a hydroxy fatty acid ester and hence is an emulsifier which includes a fatty acid esterified with a hydroxyl group, wherein the fatty acid has at least one carbon chain consisting of 18 or fewer carbon atoms. It has refatting and moisturizing properties and is highly suitable as an emulsifier for oil-in-water emulsions. In addition, it has skincare properties. Polyglyceryl-3 Caprate includes a derivative of capric acid, i.e. a saturated fatty acid the fatty acid moiety of which has a carbon chain consisting of 10 carbon atoms.

The positive effect that results from the use of an emulsifier which includes a fatty acid esterified with a hydroxyl group, wherein the fatty acid has at least one carbon chain consisting of 18, 16 or 12 carbon atoms (examples 1, 2 or 3), is additionally amplified by the use of a corresponding emulsifier having a carbon chain consisting of 10 carbon atoms.

Caprylic/Capric Triglyceride is an emulsifier from the group of polymers. If the emulsifier is selected from the group of polymers, the growth of the microorganisms on the skin is additionally inhibited.

The composition according to example 4 includes Cetearyl Glucoside as a further emulsifier. Cetearyl Glucoside is a glucoside which has been etherified with alcohols having carbon chains of 16 carbon atoms and with alcohols having carbon chains of 18 carbon atoms. If the emulsifier is an ether, a polymer, an amide, an amine or a sulfate, excessive growth of the microorganisms on the skin is prevented. A composition which includes at least one emulsifier selected from the group comprising ethers, polymers, amides, amines and sulfates only interacts with the skin flora to a minor extent, if at all. Cetearyl Glucoside is additionally used to improve the uniform distribution of skincare products when applied and enables the formation of finely dispersed mixtures of oil and water.

Like all glycols, Pentylene Glycol is derived from ethylene glycol. Moisturizing factors such as Sodium Lactate and Ectoin are substances which act as emollients in the skin. Sodium Lactate regulates the physiological pH together with other substances as constituent of the skin's own acid mantle. The slightly acidic pH of the acid mantle keeps the microbial composition of the skin in physiological equilibrium. Glycerol (Glycerin) has primarily water-binding properties.

Panthenol has skincare properties. It increases the moisture-retention capacity and improves the elasticity of the skin by increasing the rate of lipid synthesis. It also promotes the new formation of skin cells and thus contributes to regeneration. Furthermore, panthenol also has itch-relieving and anti-inflammatory properties and properties promoting wound-healing.

Xanthan gum is a naturally occurring polysaccharide which promotes the imparting of consistency to a composition and improves the product stability.

Octyldodecanol is an organic compound from the substance class of the alcohols and is used as an additive for the masking of odors and as a fragrance.

To produce the emulsion shown by way of example in table 4, first of all the substances of phase A are dissolved or mixed in the sequence specified. Next, or in parallel therewith, phase B is dissolved or mixed in the sequence specified and added to phase A. This is followed by setting the pH to 5.3 to 5.5 using lactic acid (phase C).

The mixture of phases A, B and C is homogenized at approximately 12 000 rpm (revolutions per minute). Lastly, the emulsion is stirred until smooth. The final composition is consequently a homogeneous emulsion of phases A, B and C.

All steps for producing the emulsion according to example 1 are preferably carried out at room temperature.

### Exemplary Embodiment 5

According to a fifth exemplary embodiment, a composition can be provided in the form of a washing foam. The composition according to exemplary embodiment 5 contains the substances specified in table 5.

TABLE 5
Ingredients of a washing foam
according to the invention.

| Phase | INCI nomenclature | Amount [%] |
|---|---|---|
| A | Aqua dest. | 67.00 |
| | Amphoacetate | 8.00 |
| | Pentylene Glycol | 5.00 |
| | Ethylhexylglycerin | 0.50 |
| | Sodium Lactate | 0.10 |
| | Glycerin | 4.00 |
| | Ectoin | 0.20 |
| | Dexpanthenol | 0.20 |
| B | Lactic Acid pH 5.3-5.5 | q.s. |

In the present exemplary embodiment, the emulsifier used is an amphoacetate, and therefore an amide.

Amphoacetates are amides the carbon chains of which have at least 8 carbon atoms. According to exemplary embodiment 5, it is provided that the carbon chain consists of 17 or 18. The amphoacetate used is furthermore a relatively mild emulsifier which interacts with the skin flora only to a minor extent, if at all. It stabilizes foaming compositions, in that it increases the surface tension of the liquid surrounding the individual foam bubbles.

It has surprisingly been found that a composition which contains at least one emulsifier which is an amide, an amine or a sulfate interacts with the skin flora only to a minor degree, if at all. This prevents excessive growth of individual pathogens, as a result of which disruption to the equilibrium of the microflora on the skin can be counteracted.

Pentylene Glycol, Ethylhexylglycerin, Sodium Lactate, glycerol (Glycerin) and Ectoin are humectants. Besides moisture-binding properties, they also possess antibacterial or antimicrobial properties. Like all glycols, Pentylene Glycol is derived from ethylene glycol. Moisturizing factors such as Sodium Lactate and Ectoin are substances which act as emollients in the skin. Sodium Lactate regulates the physiological pH together with other substances as constituent of the skin's own acid mantle. The slightly acidic pH of the acid mantle keeps the microbial composition of the skin in physiological equilibrium. Glycerol primarily has water-binding properties.

Dexpanthenol has skincare properties. It increases the moisture-retention capacity and improves the elasticity of the skin by increasing the rate of lipid synthesis. It also promotes the new formation of skin cells and thus contributes to regeneration. Furthermore, dexpanthenol also has itch-relieving and anti-inflammatory properties and properties promoting wound-healing.

To produce the composition shown by way of example in table 5, the listed substances of phase A are mixed together in the sequence specified. The pH of the washing foam is then set to 5.3 to 5.5 using lactic acid (Lactic Acid, phase B). The final composition is a homogeneous mixture of phases A and B.

### Exemplary Embodiment 6

According to a sixth exemplary embodiment, a further composition can be provided in the form of a washing foam. The composition according to exemplary embodiment 6 contains the substances specified in table 6 and is produced in an analogous manner to exemplary embodiment 5.

TABLE 6
Ingredients of a washing foam
according to the invention.

| Phase | INCI nomenclature | Amount [%] |
|---|---|---|
| A | Aqua dest. | 67.00 |
| | Sodium Cocoamphoacetate WAS 32% | 8.00 |
| | Pentylene Glycol | 5.00 |
| | Ethylhexylglycerin | 0.50 |
| | Sodium Lactate | 0.10 |
| | Glycerol | 4.00 |
| | Ectoin | 0.20 |
| | Dexpanthenol | 0.20 |
| B | Lactic Acid pH 5.3-5.5 | q.s. |

In the present exemplary embodiment, Sodium Cocoamphoacetate WAS 32% is used as emulsifier.

Sodium Cocoamphoacetate WAS 32% is an amphoacetate and hence an amide. The carbon chain of Sodium Cocoamphoacetate WAS 32% has 12 carbon atoms. Sodium Cocoamphoacetate WAS 32% is also a relatively mild emulsifier which only interacts with the skin flora to a minor degree, if at all. It stabilizes foaming compositions, in that it increases the surface tension of the liquid surrounding the individual foam bubbles.

It has surprisingly been found that a composition which contains at least one emulsifier which is an amide, an amine or a sulfate interacts with the skin flora only to a minor degree, if at all. This prevents excessive growth of individual pathogens, as a result of which disruption to the equilibrium of the microflora on the skin can be counteracted.

The positive effect according to which excess growth of individual pathogens on the skin is prevented, as a result of which in turn disruption to the equilibrium of the microflora on the skin can be counteracted, is additionally amplified by use of a composition comprising Sodium Cocoamphoacetate WAS 32% as emulsifier.

The washing foam according to exemplary embodiment 6 is produced in an analogous manner to the washing foam according to exemplary embodiment 5.

### Exemplary Embodiment 7

According to a seventh exemplary embodiment, a further composition can be provided in the form of a washing foam. The composition according to exemplary embodiment 7 contains the substances specified in table 7.

TABLE 7
Ingredients of a washing foam
according to the invention.

| Phase | INCI nomenclature | Amount [%] |
|---|---|---|
| A | Aqua dest. | 67.00 |
| | Sodium Cocoamphoacetate WAS 32% | 8.00 |
| | Cocamidopropyl Betaine WAS 48% | 15.00 |
| | Pentylene Glycol | 5.00 |
| | Ethylhexylglycerin | 0.50 |
| | Sodium Lactate | 0.10 |
| | Glycerol | 4.00 |
| | Ectoin | 0.20 |
| | Dexpanthenol | 0.20 |
| B | Lactic Acid pH 5.3-5.5 | q.s. |

In addition to Sodium Cocoamphoacetate WAS 32%, Cocamidopropyl Betaine WAS 48% is also used as emulsifier here.

Cocamidopropyl Betaine WAS 48% (CAPB) is an emulsifier selected from the group comprising amides and amines. It exhibits amphoteric behavior in neutral solutions, and thus exhibits different material properties depending on the reaction partner. For example, CAPB behaves as a cationic emulsifier in acidic solutions and in alkaline solutions behaves as an anionic emulsifier. CAPB additionally regulates viscosity and ensures uniform distribution of the composition when applied to the skin. It has cleansing and antistatic effects, in that the electric charge at the skin surface is neutralized. It can additionally be used to improve the foam quality of the composition. Moreover, CAPB has antiseptic properties.

It has been found that a composition which contains at least one emulsifier selected from the group comprising amides and amines only interacts with the skin flora to a minor extent, if at all. This prevents excessive growth of individual pathogens, as a result of which disruption to the equilibrium of the microflora on the skin can be counteracted.

To produce the washing foam shown by way of example in table 7, the listed substances of phase A are mixed together in the sequence specified. The pH of the washing foam is then set to 5.3 to 5.5 using lactic acid (Lactic Acid, phase B). The final composition is accordingly a homogeneous washing foam formed of phases A and B.

### Exemplary Embodiment 8

According to an eighth exemplary embodiment, a composition can be provided in the form of a water-in-oil emulsion. The composition according to exemplary embodiment 8 contains the substances specified in table 8.

All steps for producing the water-in-oil emulsion according to example 8 are preferably carried out at room temperature.

TABLE 8
Ingredients of a water-in-oil emulsion
according to the invention.

| Phase | INCI nomenclature | Amount [%] |
|---|---|---|
| A | Aqua dest. | 69.40 |
| | Pentylene Glycol | 4.00 |
| | Glycerin | 2.50 |
| | Panthenol | 1.35 |
| | Sodium Lactate | 0.20 |
| | Ectoin | 1.00 |
| | Magnesium Sulfate Heptahydrate | 0.80 |
| C | Lactic Acid | q.s |
| B | Caprylic/Capric Triglyceride | 8.00 |
| | Tocopheryl Acetate | 0.50 |
| | Tocopherol | 0.05 |
| | Cetyl PEG/PPG-10/1 Dimethicone | 2.00 |
| | Acrylates/C10-30 Alkyl Acrylate | 0.20 |
| | Crosspolymer | |
| | Octyldodecanol | 10.00 |

Magnesium Sulfate Heptahydrate is an emulsifier from the group of sulfates. This emulsifier generates lather and has advantageous thickening and conditioning properties. Magnesium Sulfate Heptahydrate is a suitable replacement for anionic surfactants.

Caprylic/Capric Triglyceride, Cetyl PEG/PPG-10/1/Dimethicone and Acrylates/C10-30 Alkyl Acrylate Crosspolymer are among the emulsifiers from the group of polymers. Dimethicone also protects the skin from external influences and thus keeps it in a smooth and supple condition. Cetyl PEG/PPG-10/1/Dimethicone is a copolymer of cetyl dimethicone (polydimethylsiloxane) and alkoxylated dimethicone.

If the emulsifier is an ether, a polymer, an amide, an amine or a sulfate, the excessive growth of the microorganisms on the skin is surprisingly prevented. A composition which includes at least one emulsifier selected from the group comprising ethers, polymers, amides, amines and sulfates only interacts with the skin flora to a minor extent, if at all. Disruption to the equilibrium of the microflora on the skin is thus counteracted.

In addition, Caprylic/Capric Triglyceride serves to improve the uniform distribution of skincare products when applied. This substance was investigated in terms of its influence on the growth of the organisms of the skin flora. No growth-promoting influence was detected. Caprylic/Capric Triglyceride is therefore suitable as an emulsifier in the composition according to the invention.

It is conceivable that the water-in-oil emulsion according to exemplary embodiment 8, instead of the emulsifiers specified or in addition thereto, contains an emulsifier selected from the group comprising ethers, amides and amines.

Panthenol has skincare properties. It increases the moisture-retention capacity and improves the elasticity of the skin by increasing the rate of lipid synthesis. It also promotes the new formation of skin cells and thus contributes to regeneration. Furthermore, panthenol also has itch-relieving and anti-inflammatory properties and properties promoting wound-healing.

Tocopheryl Acetate and Tocopherol are antioxidants. Tocopheryl Acetate is an acetic ester of vitamin E. It inhibits oxygen-caused oxidation processes and hence the degradation and deterioration of other ingredients of the composition. Tocopherol, also a derivative of vitamin E, is an umbrella term for all tocopherols having vitamin E activity. Vitamin E is a member of the fat-soluble vitamins. It builds up skin protection and promotes the regeneration of the skin.

Octyldodecanol is an organic compound from the substance class of the alcohols and is used as an additive for the masking of odors and as a fragrance.

To produce the water-in-oil emulsion shown by way of example in table 8, first of all the substances of phase A are dissolved or mixed in the sequence specified. Next, the pH of phase A is set to 5.3 to 5.5 using lactic acid (phase C). Following this or in parallel therewith, phase B is dissolved or mixed in the sequence specified and added to the mixture of phases A and C.

The mixture of phases A, B and C is homogenized at approximately 12 000 rpm (revolutions per minute). Lastly, the emulsion is stirred until smooth. The final composition is consequently a homogeneous water-in-oil emulsion of phases A, B and C.

All steps for producing the water-in-oil emulsion according to example 5 are preferably carried out at room temperature.

### Exemplary Embodiment 9

According to a ninth exemplary embodiment, a composition can be provided in the form of an oil-in-water emulsion. The composition according to exemplary embodiment 9 contains the substances specified in table 9.

TABLE 9
Ingredients of an oil-in-water emulsion
according to the invention

| Phase | INCI nomenclature | Amount [%] |
|---|---|---|
| A | Aqua Dest. | 70.00 |
| | Pentylene Glycol | 5.00 |
| | Magnesium Sulfate Heptahydrate | 0.10 |
| | Glycerin | 2.50 |
| | Panthenol | 1.35 |
| | Sodium Lactate | 0.10 |
| | Ectoin | 1.00 |
| B | Tocopheryl Acetate | 0.20 |
| | Tocopherol | 0.05 |
| | Hydroxyethyl Acrylate/Sodium | 1.20 |
| | Acryloyldimethyl Taurate | |
| | Copolymer | |
| | Octyldodecanol | 10.00 |
| | Caprylic/Capric Triglyceride | 8.50 |
| C | Lactic Acid | q.s |

Magnesium Sulfate Heptahydrate is an emulsifier from the group of sulfates. This emulsifier generates lather and has advantageous thickening and conditioning properties. Magnesium Sulfate Heptahydrate is a suitable replacement for anionic surfactants.

Caprylic/Capric Triglyceride, Cetyl PEG/PPG-10/1/Dimethicone and Acrylates/C10-30 Alkyl Acrylate Crosspolymer are among the emulsifiers from the group of polymers. Dimethicone also protects the skin from external influences and thus keeps it in a smooth and supple condition. Cetyl PEG/PPG-10/1/Dimethicone is a copolymer of cetyl dimethicone (polydimethylsiloxane) and alkoxylated dimethicone.

It is conceivable that the oil-in-water emulsion according to exemplary embodiment 9, instead of the emulsifiers specified or in addition thereto, contains an emulsifier selected from the group comprising ethers, amides and amines.

If the emulsifier is an ether, a polymer, an amide, an amine or a sulfate, the rate of growth of the microorganisms on the skin surprisingly decreases. A composition which contains at least one emulsifier selected from the group comprising ethers, polymers, amides, amines and sulfates only interacts with the skin flora to a minor extent, if at all. Disruption to the equilibrium of the microflora on the skin is thus counteracted.

Hydroxyethyl Acrylate/Sodium Acryloyldimethyl Taurate Copolymer is a consistency regulator. Acrylates are polymers or copolymers based on acrylic acid or other acrylic esters. Taurates are N-acyl derivatives, esters or salts of taurine. The term copolymers refers to polymers composed of two or more different base units. Consistency regulators increase or reduce the viscosity of the composition.

Panthenol has skincare properties. It increases the moisture-retention capacity and improves the elasticity of the skin by increasing the rate of lipid synthesis. It also promotes the new formation of skin cells and thus contributes to regeneration. Furthermore, panthenol also has itch-relieving and anti-inflammatory properties and properties promoting wound-healing.

To produce the oil-in-water emulsion shown by way of example in table 9, first of all the substances of phase A are dissolved or mixed at 50° C. in the sequence specified. Following this or in parallel therewith, phase B is dissolved or mixed in the sequence specified likewise at 50° C.

Phases A and B are then homogenized at approximately 12 000 rpm (revolutions per minute). After this, the homogeneous emulsion is stirred until it has reached room temperature. The pH of the mixture of phases A and B is set to 5.3 to 5.5 using lactic acid (phase C).

### Exemplary Embodiment 10

According to a tenth exemplary embodiment, a composition can be provided in the form of an alternative oil-in-water emulsion. The composition according to exemplary embodiment 10 contains the substances specified in table 10.

TABLE 10
Ingredients of an alternative oil-in-water
emulsion according to the invention

| Phase | INCI nomenclature | Amount [%] |
|---|---|---|
| A | Aqua Dest. | 70.00 |
| | Pentylene Glycol | 5.00 |
| | Magnesium Sulfate Heptahydrate | 0.10 |
| | Glycerin | 2.50 |
| | Panthenol | 1.35 |
| | Sodium Lactate | 0.10 |
| | Ectoin | 1.00 |
| B | Tocopheryl Acetate | 0.20 |
| | Tocopherol | 0.05 |
| | Acrylates/C10-30 Alkyl Acrylate | 0.7 |
| | Crosspolymer | |
| | Cetearyl Glucoside | 0.5 |
| | Octyldodecanol | 8.00 |
| | Caprylic/Capric Triglyceride | 10.50 |
| C | Lactic Acid | q.s |

In an analogous manner to exemplary embodiment 9, the oil-in-water emulsion according to exemplary embodiment 10 contains Caprylic/Capric Triglyceride and Magnesium Sulfate Heptahydrate as emulsifiers. The oil-in-water emulsion according to exemplary embodiment 10 also additionally includes Acrylates/C10-30 Alkyl Acrylate Crosspolymer and Cetearyl Glucoside as emulsifiers.

Acrylates/C10-30 Alkyl Acrylate Crosspolymer is an emulsifier from the group of polymers. Cetearyl Glucoside is a glucoside which has been etherified with alcohols comprising carbon chains of 16 carbon atoms and with alcohols comprising carbon chains of 18 carbon atoms. It is used to improve the uniform distribution of skincare products when applied and enables the formation of finely dispersed mixtures of oil and water.

It is conceivable that the oil-in-water emulsion according to exemplary embodiment 10, instead of the emulsifiers specified or in addition thereto, contains an emulsifier selected from the group comprising ethers, amides and amines.

It has surprisingly been found that a composition which contains at least one emulsifier, which is selected from the group comprising amides, amines, polymers and sulfates and/or which includes a fatty acid which is esterified with a hydroxyl group, wherein the fatty acid has at least one carbon chain consisting of 18 or fewer carbon atoms, interacts with the skin flora only to a minor degree, if at all. In this way, excessive growth of individual organisms is prevented, as a result of which disruption to the equilibrium of the microflora on the skin can be counteracted. Moreover, the fatty acid fraction is insoluble and can precipitate out, which in the case of large molecules can in turn lead to clogging of the skin pores. For large fatty acid molecules (more than 18 carbon atoms in a carbon chain), the risk of clogging skin pores thus also increases.

Instead of Hydroxyethyl Acrylate/Sodium Acryloyldimethyl Taurate Copolymer, in exemplary embodiment 10 Acrylates/C10-30 Alkyl Acrylate Crosspolymer is present as consistency regulator.

The oil-in-water emulsion according to exemplary embodiment 10 is produced in a manner analogous to the production according to exemplary embodiment 9: First of all, the substances of phase A are dissolved or mixed at 50° C. in the sequence specified. Following this or in parallel therewith, phase B is dissolved or mixed in the sequence specified likewise at 50° C.

Phases A and B are then homogenized at approximately 12 000 rpm (revolutions per minute). After this, the homogeneous emulsion is stirred until it has reached room temperature. The pH of the mixture of phases A and B is set to 5.3 to 5.5 using lactic acid (phase C).

Table 11 summarizes the influence of some exemplary embodiments on the growth of various Malassezia strains of the skin flora. The results shown were obtained using growth analyses of the relevant microorganisms on deficient agar or by analyzing breakdown products by means of HPTLC (high-performance thin-layer chromatography). The evaluation of the growth in the case of Malassezia species can be assessed visually.

The following legend applies for table 11:

TABLE 11
Influence of the cosmetic products on
the growth of Malassezia species

| | W/O | O/W | | | |
|---|---|---|---|---|---|
| | emulsion | emulsion | Growth | W/O | O/W |
| | (ex. 5) | (ex. 6 + 7) | (ex. 1-4) | negative | negative |
| Furfur | (1−) | (1−) | (1−) | (2+) | (2+) |
| 1878 | | | | | |
| Furfur | (1−) | (1−) | (1−) | (3+) | (2+) |
| 7019 | | | | | |
| Sympodialis | (1−) | (1−) | (1−) | (1+) | (2+) |
| 7222 | | | | | |
| Sympodialis | (1−) | (1−) | (1−) | (1+) | (2+) |
| 7977 | | | | | |
| Sympodialis | (1−) | (1−) | (1−) | (1+) | (2+) |
| 42312 | | | | | |
| Sympodialis | (1−) | (1−) | (1−) | (1+) | (2+) |
| 6171 | | | | | |
| Globosa | (1−) | (1−) | (1−) | (1+) | (1+) |
| 7966 | | | | | |
| Globosa | (1−) | (1−) | (1−) | (1+) | (1+) |
| 7966 | | | | | |
| Globosa | (1−) | (1−) | (1−) | (1+) | (1+) |
| PM1 | | | | | |
| Globosa | (1−) | (1−) | (1−) | (1+) | (1+) |
| 7705 | | | | | |
| Restricta | (1−) | (1−) | (1−) | (1+) | (1+) |
| 7877 | | | | | |

(1−) = no growth;
(1+) = slight growth;
(2+) = heavy growth;
(3+) = very heavy growth.

The influence of the exemplary embodiments on the growth of various bacterial strains of the skin flora is summarized in table 12. The results shown were obtained using growth analyses of the relevant microorganisms on deficient agar or by analyzing breakdown products by means of HPTLC (high-performance thin-layer chromatography).

The following legend applies for table 12:

TABLE 12
Influence of the cosmetic products
on the growth of bacteria

| | W/O | O/W | |
|---|---|---|---|
| | emulsion | emulsion | Washing foam |
| | (ex. 5) | (ex. 6 + 7) | (ex. 1-4) |
| Staphylococcus | ∘ | ∘ | ∘ |
| epidermidis | | | |
| Staphylococcus | ∘ | ∘ | ∘ |
| galinarum | | | |
| Corynebacterium | ∘ | ∘ | ∘ |
| minutissimum | | | |
| Streptococcus | ∘ | ∘ | ∘ |
| mitis | | | |
| Propionibacterium | ∘ | ∘ | ∘ |
| acnes | | | |

(−) = reduced growth;
(+) = growth;
(∘) = no influence.

The invention is not restricted to the embodiments described above and instead can be modified in a multitude of ways.

All features and advantages arising from the claims, the description and the drawing, including chemical process steps, may be essential to the invention both taken alone and in a very wide variety of combinations.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A composition for topical application to the skin, comprising:
at least one humectant,
at least one consistency regulator, and
at least one emulsifier,
wherein the emulsifier includes a fatty acid esterified with a hydroxyl group, wherein the fatty acid has at least one carbon chain of 18 or fewer carbon atoms.

2. The composition as claimed in claim 1, wherein the emulsifier includes a fatty acid esterified with a hydroxyl group, wherein the fatty acid has at least one carbon chain of 16 or fewer carbon atoms.

3. The composition as claimed in claim 2, wherein the emulsifier includes a fatty acid esterified with a hydroxyl group, wherein the fatty acid has at least one carbon chain of 12 or fewer carbon atoms.

4. The composition as claimed claim 1, wherein the at least one emulsifier is selected from the group consisting of hydroxy fatty acid esters, sugar esters, and polyglycerol esters.

5. A composition for topical application to the akin, comprising:
at least one humectant,
at least one consistency regulator, and
at least one emulsifier,
wherein the emulsifier is selected from the group comprising ethers, polymers, amides, amines and sulfates.

6. The composition as claimed in claim 5, wherein the at least one emulsifier is an ether, selected from the group comprising PEG ethers and alkyl polyglycosides.

7. The composition as claimed in claim 5, wherein the at least one emulsifier is a polymer selected from the group consisting of:
Cetyl PEG/PPG-10/1/Dimethicone,
Bis-PEG/PPG-16/16 PEG/PPG-16/16 Dimethicone,
Bis-PEG/PPG-20/5 PEG/PPG-20/5 Dimethicone,
Methoxy PEG/PPG-25/4 Dimethicone,
Poloxamer 407, and
Acrylates/C10-30 Alkyl Acrylate Crosspolymer.

8. The composition as claimed in claim 5, wherein the at least one emulsifier is an amide or an amine selected from the group consisting of:
Capryl/Capramidopropyl Betaine,
Cocamidopropyl Betaine WAS 48%,
Cocamidopropyl Betaine,
Undecylenamidopropyl Betaine,
Disodium Cocoamphodiacetate,
Palmitamidopropyltrimonium Chloride,
Distearyldimonium Chloride, and
Disodium/Sodium Cocoyl Glutamate.

9. The composition as claimed in claim 5, wherein the at least one emulsifier is a sulfate selected from the group consisting of Sodium Coco-Sulfate and Sodium Laureth Sulfate.

10. A composition for topical application to the skin, comprising:
at least one humectant,
at least one consistency regulator, and
at least one emulsifier,
wherein the emulsifier is selected from the group consisting of ethers, polymers, amides, amines, and sulfates, and
wherein the emulsifier includes a fatty acid esterified with a hydroxyl group, wherein the fatty acid has at least one carbon chain of 18 or fewer carbon atoms.

11. The composition as claimed in claim 10, wherein the consistency regulator is selected from the group consisting of:
xanthan gum,
cellulose derivatives,
carbomers,
Acrylamide/Sodium Acryloyldimethyl Taurate Copolymer,
Hydroxyethyl Acrylate/Sodium Acryloyldimethyl Taurate Copolymer,
Sodium Acrylate/Acryloyldimethyltaurate/Dimethylacrylamide Crosspolymer,
Hydroxyethyl Acrylate/Sodium Acryloyldimethyl Taurate Copolymer & Polyisobutene & PEG-7 Trimethylolpropane Coconut Ether,
Palmitamidopropyltrimonium Chloride,
Sodium Acrylate/Acryloyldimethyltaurate/Dimethylacrylamide Crosspolymer,
Cocamide,
capric acid,
Palmitamidopropyltrimonium Chloride,
Sorbitan Sesquicaprylate,
Isostearamide MIPA,
Glyceryl Laurate,
Laureth-4, and
fatty alcohols having at least one carbon chain of 12 or fewer carbon atoms.

12. The composition as claimed in claim 10, wherein composition comprises at least one emollient selected from the group consisting of:
Paraffin,
Polyisobutene,
squalane,
silicone oil,
Cetyl Dimethicone,
Stearyl Dimethicone,
squalene,
Cyclopentasiloxane,
Octyldodecanol, and
Caprylic/Capric Triglyceride.

13. The composition as claimed in claim 12, wherein the at least one emollient has a fatty acid moiety, wherein the carbon chain of the fatty acid moiety comprises 18 or fewer carbon atoms.

14. The composition as claimed in claim 10, further comprising at least one solubilizer selected from the group of polyethylene glycols.

15. The composition as claimed in claim 10, wherein the humectant is selected from the group consisting of glycols and polyhydric alcohols and moisturizing factors.

16. The composition as claimed in claim 10, wherein the composition is a cream, an ointment, a foam, a lotion or a gel.
